(19) United States
(12) Patent Application Publication
(10) Pub. No.: US 2025/0009974 A1
(43) Pub. Date: Jan. 9, 2025

(54) Vascular Access Device With Effective Needle Length Adjustment

(71) Applicant: BECTON, DICKINSON AND COMPANY, Franklin Lakers, NJ (US)

(72) Inventors: Sajayesh Vijayachandran, Kerala (IN); Sudev GS, Kerala (IN)

(21) Appl. No.: 18/708,733

(22) PCT Filed: Nov. 11, 2021
(86) PCT No.: PCT/US2021/058898

§ 371 Date: May 9, 2024

(51) Int. Cl. A61M 5/31; A61M 5/46; A61M 39/02

(52) U.S. Cl. CPC A61M 5/3134; A61M 5/46; A61M 39/0208

(57) ABSTRACT

Vascular access devices which have needle-length adjustment features for penetrating a vial of a stopper with the full needle length and for penetrating the skin of a patient with a desired needle length, which is less than the full needle length.

### TECHNICAL FIELD

The present disclosure relates to needle-length adjustable vascular access devices, and in particular the present disclosure relates to insulin syringes having needle length adjustment for penetrating both insulin vials and the skin of a patient.

### BACKGROUND

Syringes are commonly used in the art to withdraw medicament from a vial and injecting the medicament into the skin of a patient. Needle cannulas for syringes come in varying needle gauge and needle lengths depending on the intended use, the insertion depth into the skin of the patient and other factors. When withdrawing medicament from a vial, a thicker needle gauge can be used. Syringes can have an integrated, non-removable needle cannula affixed to the distal end of the barrel, or they can have a needleless connector which can attach to removable and interchangeable needle hubs.

Insulin syringes common in the art have a standard needle cannula length of 6 mm. The needle cannula of an insulin syringe can either be non-removably affixed to the syringe or can be attachable via a needle hub to a needleless connector of the insulin syringe. The 6 mm needle cannula length is used because a 6 mm needle cannula can penetrate a stopper of a standard insulin vial at any angle and reach the insulin in the vial for aspiration of insulin.

Insulin is commonly injected in the subcutaneous layer underneath the skin to ensure quick and uniform dispersion of insulin in the patient's body. Needle cannulas having a 6 mm length can pass through the subcutaneous layer into the muscle layer underneath it which is not desirable. To avoid this occurrence, a practitioner administering the insulin shot commonly has to pinch the site where the insulin shot/injection is to be taken to avoid the 6 mm needle cannula from penetrating into the muscle layer. This method can be a tedious process which can lead to patient discomfort, needlestick injuries and inconsistent results. In the alternative, interchangeable needle hubs can be used for first using a 6 mm needle cannula to withdraw medicament from a vial and subsequently using a smaller needle length to administer the medicament. This alternative results in further waste.

Thus, there is a need in the art to provide a single-use needle vascular access device which can adequately withdraw medicament from a vial and subsequently administer the medicament to the subcutaneous layer underneath the skin.

### SUMMARY

A first aspect of the present disclosure relates to a vascular access device having a cylindrical base, a flange and a needle cannula. The cylindrical base has a proximal end and a distal end. The flange comprises two prongs extending distally from the flange, the two prongs having a distal end extending a length from the flange, the two prongs are placed a distance Dp from each other, the distance Dp being greater than a width Wv of a rubber stopper of a vial such that the two prongs can pass over the rubber stopper. The vascular access device further comprises a distal protrusion extending distally from the flange, the distal protrusion having a distal surface and an aperture extending from the proximal end of the base to the distal surface of the distal protrusion. The needle cannula extends through the aperture and extends a distance Dc from the distal surface of the distal protrusion. The two prongs extend a distance Dn from the distal surface of the distal protrusion.

In some embodiments, the proximal end of the cylindrical base is removably attached or integral to a distal end of a barrel of a syringe. In some embodiments, the needle cannula is affixed to the distal end of the barrel extends from the distal end of the barrel of the syringe through the aperture.

In some embodiments, the two prongs are at a substantially right angle relative to the flange forming a U-shape with the flange.

In some embodiments, the distance Dc is sufficient to penetrate the rubber stopper of the vial. In some embodiments, the distance Dc is 6 mm and the vial is an insulin vial. In some embodiments, the needle cannula is sufficient to penetrate the insulin vial at an angle having a range between 90 to 45 degrees relative to the rubber stopper.

In some embodiments, the prongs are effective to limit the needle cannula from fully entering a skin of a patient by the distance Dn. In some embodiments, the distance Dc of the needle cannula is 6 mm and the distance Dn is 2 mm, and where the two prongs limit contact of the distal surface of the distal end from contacting a flat surface, the cannula can only be inserted 4 mm into said flat surface.

In some embodiments, the flange is connected to the base by connection points of the flange for connecting the flange to a pivot located at the distal end of the base. In some embodiments, the base and the flange are separate components which snap-fit together by a pivot of the flange and connection points of the distal end of the base. In some embodiments, the pivot and connection points have an angular range of motion 0 degrees to 75 degrees. In some embodiments, the base and the flange are of a unitary construction and connected by a living hinge to provide a range of motion of the base of the flange relative to the base.

A second aspect of the disclosure relates to a vascular access device having a cylindrical base, a compressible foam tip and a needle cannula. The cylindrical base has a proximal end and a distal end, the distal end has an elongate tip and a collar extending therefrom, the collar surrounding the elongate tip, the elongate tip having an aperture extending from the proximal end of the base to the elongate tip, the collar spaced a distance from the elongate tip such that a channel is formed between the collar and the elongate tip, the channel having a distal surface. The compressible foam tip has a proximal end and a distal end, and a proximal portion and a distal portion, the distal portion having a larger diameter than a diameter of the proximal portion forming a contact surface, the compressible foam tip inserted and affixed to the base where the distal surface of the base abutting the contact surface of the compressible foam tip, the distal portion of the compressible foam tip is compressible from an initial uncompressed length Lu in an initial uncompressed state to a final compressed length Lc in a final compressed state; and. The needle cannula extends through the aperture and extends a distance Dc′ from the distal portion in the uncompressed state; Compression of the compressible foam tip to the final compressed state results in the needle cannula further extending by a length L in addition to the distance Dc′.

In some embodiments, the proximal end of the cylindrical base is removably attached or integral to a distal end of a barrel of a syringe. In some embodiments, the needle cannula is affixed to the distal end of the barrel extends from the distal end of the barrel of the syringe through the aperture. In some embodiments, the elongate tip and collar extend the same length and share the distal surface. In some embodiments, the base further comprises a glue channel below the distal end of the base and at least partially within the channel, the glue channel lateral to the channel. In some embodiments, the proximal end of the compressible foam tip is inserted into the glue channel, the proximal end of the compressible foam tip having a substantially equal width with the glue channel, wherein a cavity is formed between the elongate tip of the base and the proximal portion of the compressible foam tip.

In some embodiments, the compressible foam tip is compressed by applying a compression force against a top surface of a rubber stopper such that the needle cannula is inserted into the top surface of the rubber stopper by a combined length of distance Dc′ and length L.

In some embodiments, the distance Dc′ is 4 mm and the combined length of distance Dc′ and length L is 6 mm.

A third aspect of the present disclosure is directed to a vascular access device having a cylindrical base having a proximal end and a distal end, the distal end including a distal end geometry, the base including an aperture extending through the base, the distal end geometry having a sloped surface and a distally flat surface located distal to the sloped surface, the sloped surface is sloped at an angle Θ relative to the base, the distally flat surface located a distance Dn from the aperture; and, a needle cannula extending through the aperture, needle cannula extending from the sloped surface at the aperture by a distance Dc.

In some embodiments, the distally flat surface includes a lateral extension. In some embodiments, the distance Dc is 6 mm and the distance Dn is 2 mm, and the needle cannula has an effective length of 4 mm.

### BRIEF DESCRIPTION OF THE DRAWINGS

FIG. 1 illustrates a side view of a vascular access device attached to a syringe in accordance with one or more embodiments of the present disclosure;

FIGS. 2A and 2B illustrate cross-sectional views of a vascular access device penetrating a vial in accordance with one or more embodiments of the present disclosure;

FIG. 3A illustrates a cross-sectional view of a vascular access device penetrating a vial in accordance with one or more embodiments of the present disclosure;

FIG. 3B illustrates a cross-sectional view of a vascular access device penetrating a vial in accordance with one or more embodiments of the present disclosure;

FIG. 4 illustrates a cross-sectional view of a vascular access device penetrating the skin of a patient in accordance with one or more embodiments of the present disclosure;

FIG. 5 illustrates a side view of a vascular access device attached to a syringe in accordance with one or more embodiments of the present disclosure;

FIG. 6 illustrates a detailed perspective of a vascular access device attached to a syringe in accordance with one or more embodiments of the present disclosure;

FIG. 7 illustrates a detailed side view of a vascular access in accordance with one or more embodiments of the present disclosure;

FIG. 8 illustrates a cross-sectional view of a vascular access device penetrating a vial in accordance with one or more embodiments of the present disclosure;

FIG. 9 illustrates a cross-sectional view of a vascular access device penetrating a vial in accordance with one or more embodiments of the present disclosure;

FIG. 10 illustrates a cross-sectional view of a vascular access device penetrating the skin of a patient in accordance with one or more embodiments of the present disclosure;

FIG. 11 illustrates a perspective of a vascular access device attached to a syringe in accordance with one or more embodiments of the present disclosure;

FIG. 12 illustrates an exploded side view of a vascular access device attached to a syringe in accordance with one or more embodiments of the present disclosure;

FIG. 13A illustrates a detailed side view a vascular access device in accordance with one or more embodiments of the present disclosure;

FIG. 13B illustrates a detailed cross-sectional view a vascular access device in accordance with one or more embodiments of the present disclosure;

FIG. 14A illustrates a perspective view of a base of a vascular access device in accordance with one or more embodiments of the present disclosure;

FIG. 14B illustrates a cross-sectional view of a base of a vascular access device in accordance with one or more embodiments of the present disclosure;

FIG. 15A illustrates a perspective view of a foam tip of a vascular access device in accordance with one or more embodiments of the present disclosure;

FIG. 15B illustrates a cross-sectional view of a foam tip of a vascular access device in accordance with one or more embodiments of the present disclosure;

FIG. 16 illustrates a foam tip of a vascular access device in an initial, uncompressed state in accordance with one or more embodiments of the present disclosure;

FIG. 17 illustrates a foam tip of a vascular access device in a final, compressed state in accordance with one or more embodiments of the present disclosure;

FIG. 18 illustrates a foam tip of a vascular access device in a final, compressed state in accordance with one or more embodiments of the present disclosure;

FIG. 19 illustrates a foam tip of a vascular access device in an initial, uncompressed state in accordance with one or more embodiments of the present disclosure;

FIGS. 20A through 20D illustrate steps of a method of withdrawing medicament from a vial and administering the medicament into the skin of a patient in accordance with one or more embodiments of the present disclosure;

FIG. 21 illustrates a side view of a vascular access device attached to a syringe in accordance with one or more embodiments of the present disclosure;

FIG. 22 illustrates a detailed view a vascular access device attached to a syringe in accordance with one or more embodiments of the present disclosure;

FIG. 23 illustrates a cross-sectional view of a vascular access device penetrating a vial in accordance with one or more embodiments of the present disclosure;

FIG. 24 illustrates a cross-sectional view of a vascular access device penetrating a vial in accordance with one or more embodiments of the present disclosure; and,

FIG. 25 illustrates a cross-sectional view of a vascular access device penetrating the skin of a patient in accordance with one or more embodiments of the present disclosure;

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

For purposes of the description hereinafter, the terms “proximal”, “distal”, “longitudinal”, and derivatives thereof shall relate to the disclosure as it is oriented in the drawing figures. However, it is to be understood that the disclosure may assume alternative variations and step sequences, except where expressly specified to the contrary. It is also to be understood that the specific devices and processes illustrated in the attached drawings, and described in the following specification, are simply exemplary embodiments of the disclosure. Hence, specific dimensions and other physical characteristics related to the embodiments disclosed herein are not to be considered as limiting.

As used herein, the use of “a,” “an,” and “the” includes the singular and plural.

As used herein, the term “Luer connector” refers to a connection collar that is the standard way of attaching syringes, catheters, hubbed needles, IV tubes, etc. to each other. The Luer connector consists of one or more interlocking tubes, slightly tapered to hold together with just a simple pressure/twist fit/friction fit. Luer connectors can optionally include an additional outer rim of threading, allowing them to be more secure. The Luer connector can interlock and connect to the end located on the vascular access device (VAD). A Luer connector comprises a distal end, a proximal end, an irregularly shaped outer wall, a profiled center passageway for fluid communication from the chamber of the barrel of a syringe to the hub of a VAD. A Luer connector also has a distal end channel that releasably attaches the Luer connector to the hub of a VAD, and a proximal end channel that releasably attaches the Luer connector to the barrel of a syringe. As used herein, the term “Luer connector” refers to a male luer connector or a female luer connector.

As used herein, the term “medical device” refers to common medical devices having threaded or interlocking connections, the connections having corresponding mating elements. By way of example but not limitation, a syringe may have a threaded connection which releasably interlocks with a secondary medical device such as a needless connector of a catheter, an IV line and the like. The threaded connection may include a lumen defining a fluid path surrounded by a protruding wall having the threaded means for attaching to the secondary medical device.

As would be readily appreciated by skilled artisans in the relevant art, while descriptive terms such as “thread”, “taper”, “tab”, “wall”, “proximal”, “side”, “distal” and others are used throughout this specification to facilitate understanding, it is not intended to limit any components that can be used in combinations or individually to implement various aspects of the embodiments of the present disclosure.

Embodiments of the present disclosure are directed to vascular access devices which have needle-length adjustment features for penetrating a vial of a stopper with the full needle length and for penetrating the skin of a patient with a desired needle length, which is less than the full needle length. The vascular access devices described herein can be integral to a syringe barrel or can be attachable to a needless connector of a syringe barrel. The vascular access devices described herein can be integral to any medical device for withdrawing or injecting medicament from a vial and injecting into the skin of a patient. In some embodiments, the vascular access devices are either integral or removably connectable to insulin syringes.

In insulin administration, needle cannulas commonly have a length of 6 mm because such a length is capable of penetrating a stopper of a standard insulin vial at any angle and reach the insulin in the vial for aspiration of insulin. The same needle cannula used to withdraw medicament from a vial is subsequently used to inject the medicament into the skin of a patient. For insulin administration, the insulin medicament is administered in the subcutaneous layer underneath the skin to ensure quick and uniform dispersion of insulin in the patient's body. However, a cannula length of 6 mm will result in passing through the subcutaneous layer into the muscle layer underneath it which is not desirable. Stated differently, if injected, the muscle layer is perpendicular and under the subcutaneous layer. Embodiments of the present disclosure have passive features which enable a needle cannula of (by way of example but not limitation, a 6 mm needle cannula) to be fully inserted into a vial at any angle but only partially inserted into the skin of a patient at a desired skin depth (by way of example but not limitation, into the subcutaneous layer underneath the skin).

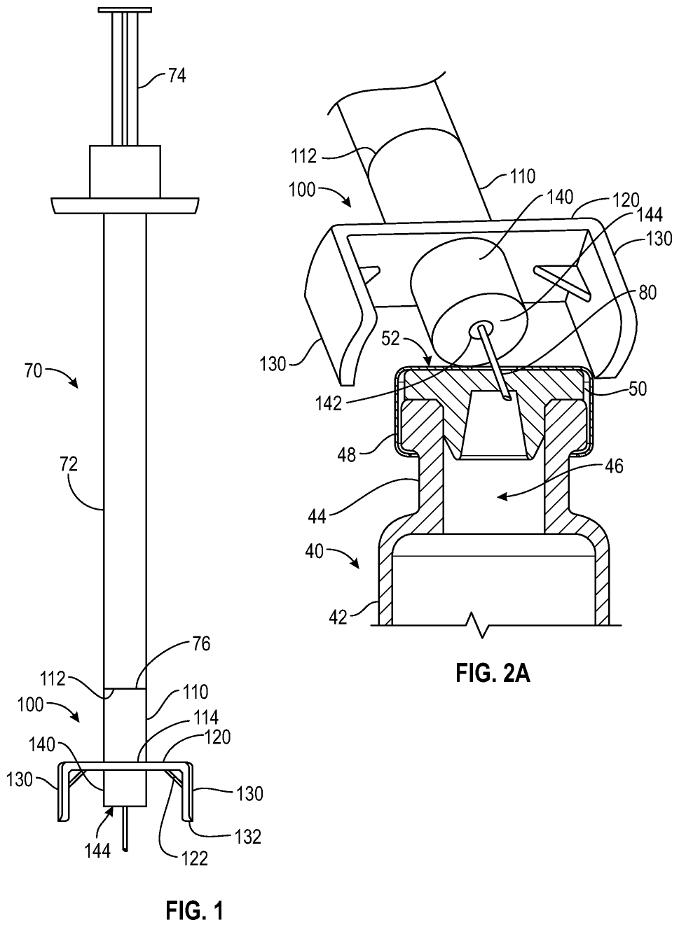

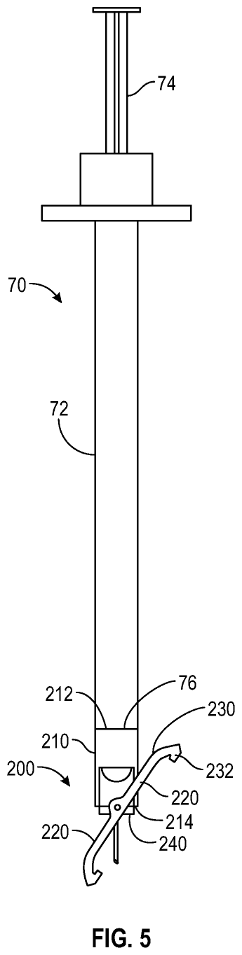

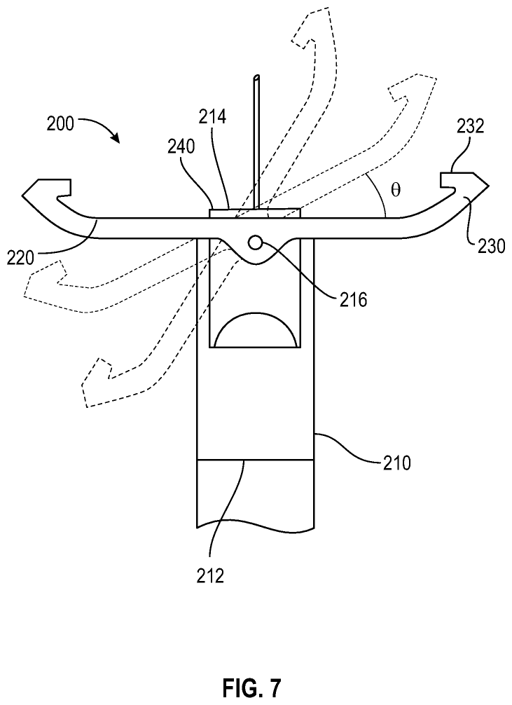

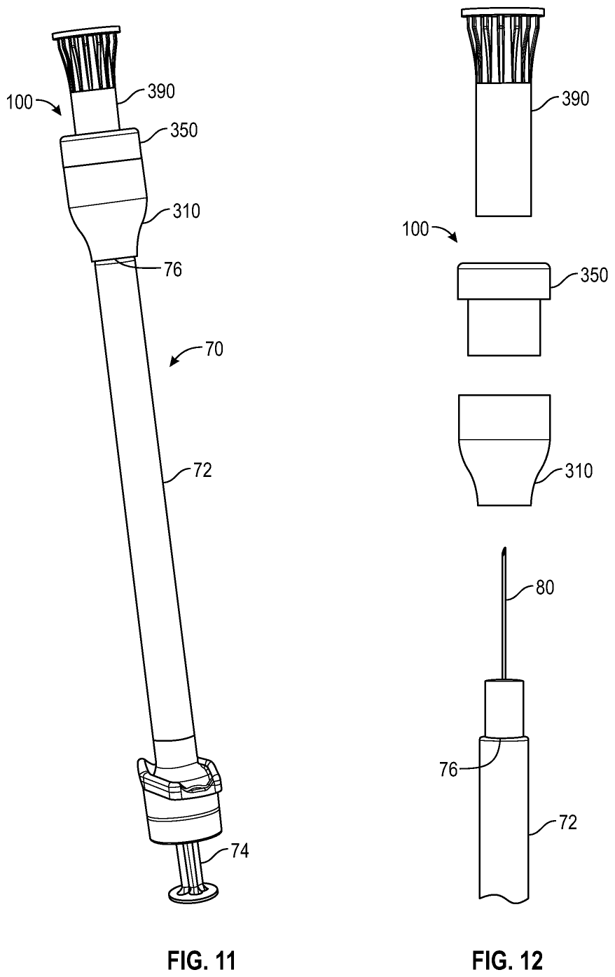

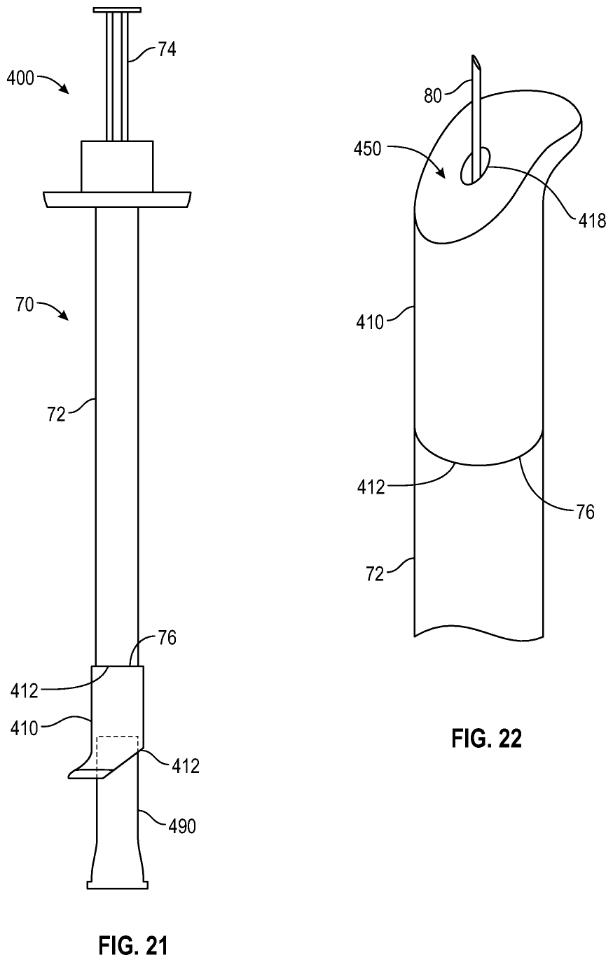

FIGS. 1-4 illustrate an embodiment of a vascular access device 100 having a fixed flange for partially inserting a fixed-length needle cannula into the skin of a patient at a desired skin depth. FIGS. 5-10 illustrate an embodiment of a vascular access device 200 having a swivel-arm flange for partially inserting a fixed-length needle cannula into the skin of a patient at a desired skin depth. FIGS. 11-20D illustrate an embodiment of a vascular access device 300 having a depressible foam tip for partially inserting a fixed-length needle cannula into the skin of a patient at a desired skin depth. FIGS. 21-25 illustrate an embodiment of a vascular access device 400 having a cammed geometry for partially inserting a fixed-length needle cannula into the skin of a patient at a desired skin depth.

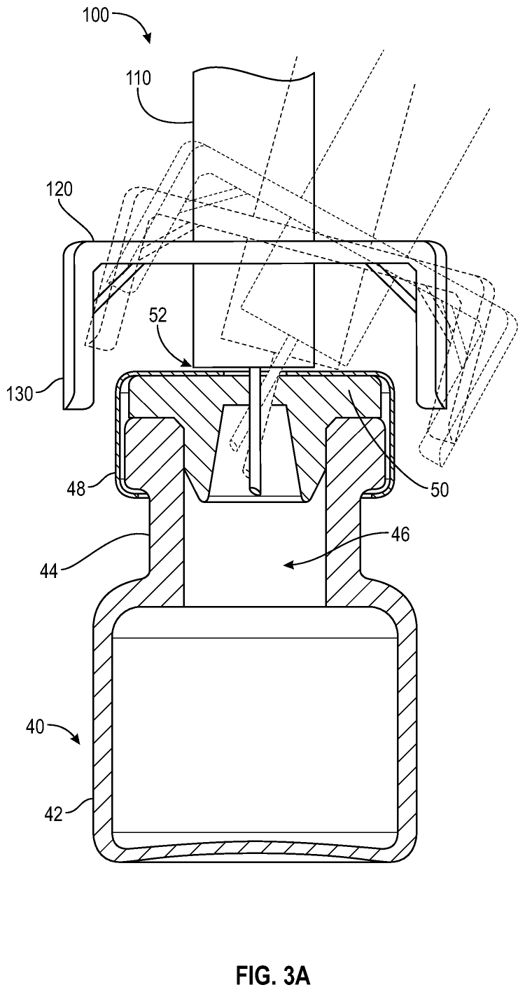

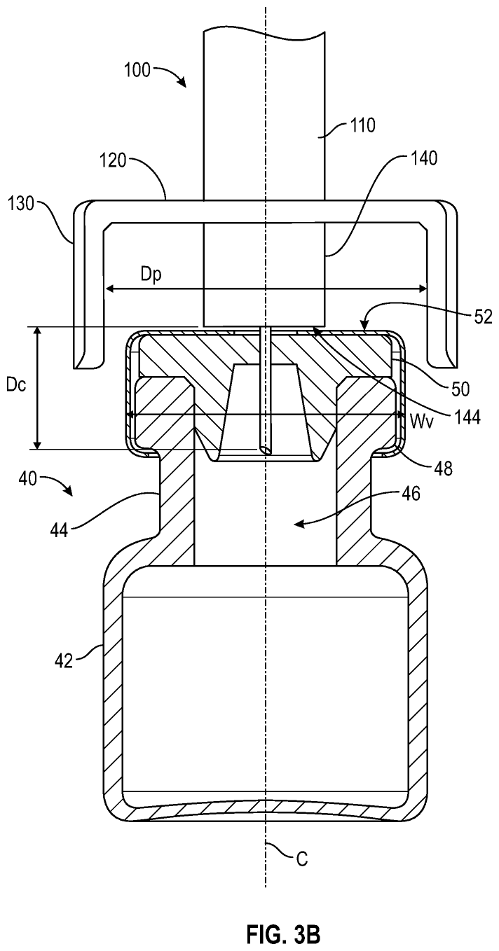

Throughout the figures (FIGS. 1-25), a conventional vial 40 is illustrated. The vial 40 has a cylindrical body 42 having a cavity for storing medicament. The cylindrical body 42 has a closed base and a neck 44 opposite the closed base. The neck 44 has an opening 46 into which a rubber stopper 50 is inserted, creating a seal. In some embodiments, the neck 44 further comprises a flange 48. In some embodiments, the rubber stopper 50 has a trapezoidal shape. In some embodiments, the rubber stopper 50 has a width substantially equal to a width of the flange 48. As shown in the figures, a needle cannula penetrates a top surface 52 of the rubber stopper 50 to withdraw medicament from the vial 40. As best shown in FIG. 3B, the rubber stopper 50 of the vial 40 a width Wv.

Throughout the figures (FIGS. 1-25), a conventional syringe 70 is illustrated. The syringe 70 includes a barrel 72 having a distal end 76. The syringe further includes a plunger rod 74 which can be partially withdrawn from the barrel 72, causing an internal negative pressure within the barrel 72 such that fluid or medicament can be drawn into the barrel 72. Likewise, the plunger rod 74 can be depressed into the barrel 72, causing an internal positive pressure buildup such that fluid or medicament can be dispelled out of the barrel 72. In some embodiments, the distal end 76 is integral to one or more of the vascular access devices (100, 200, 300, 400) as explained in further detail below. In some embodiments, the distal end 76 is removably attachable to one or more of the vascular access devices (100, 200, 300, 400) as explained in further detail below. In some embodiments, the distal end 76 is removably attachable to one or more of the vascular access devices (100, 200, 300, 400) by a needleless connection.

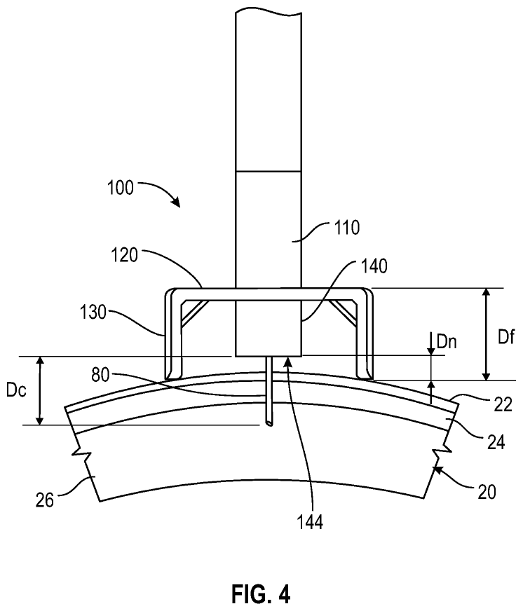

Throughout the figures (FIGS. 4, 10, and 25), a cross-section of a skin 20 of a patient is illustrated. The outermost layer is the epidermis layer 22. Under the epidermis layer 22 is the dermis layer 24 and under the dermis layer 24 is the subcutaneous layer 26. Insulin is commonly injected in the subcutaneous layer 26 underneath the skin to ensure quick and uniform dispersion of insulin in the patient's body. Needle cannulas having a 6 mm length can pass through the subcutaneous layer 26 into the muscle layer (not shown), which is not desirable.

As shown in FIGS. 1-4, the vascular access device 100 comprises a cylindrical base 110 having a proximal end 112 removably attached or integral to the distal end 76 of the syringe, and a distal end 114. An aperture 142 extends through the cylindrical base 110 configured to house the needle cannula 80.

The distal end 114 further comprises a flange 120 extending outward from the distal end 114 at a substantially right angle relative to the base 110, and is integral to the base 110. The flange 120 further comprises two prongs 130 extending distally from the flange 120, the two prongs 130 have a distal end 132 extending a length from the flange 120. The two prongs 130 are at a substantially right angle relative to the flange 120 forming a U-shape with the flange 120. In some embodiments, the flange 120 has a discoid shape, in some embodiments, the flange has a trigonal shape. In embodiments where the flange 120 has a discoid shape, the two prongs can be a uniform collar surrounding the discoid shaped flange 120. In some embodiments, a rib 122 extends between the flange 120 and each of the two prongs 130. The rib 122 is configured to provide additional structural rigidity to the flange 120 and prongs 130.

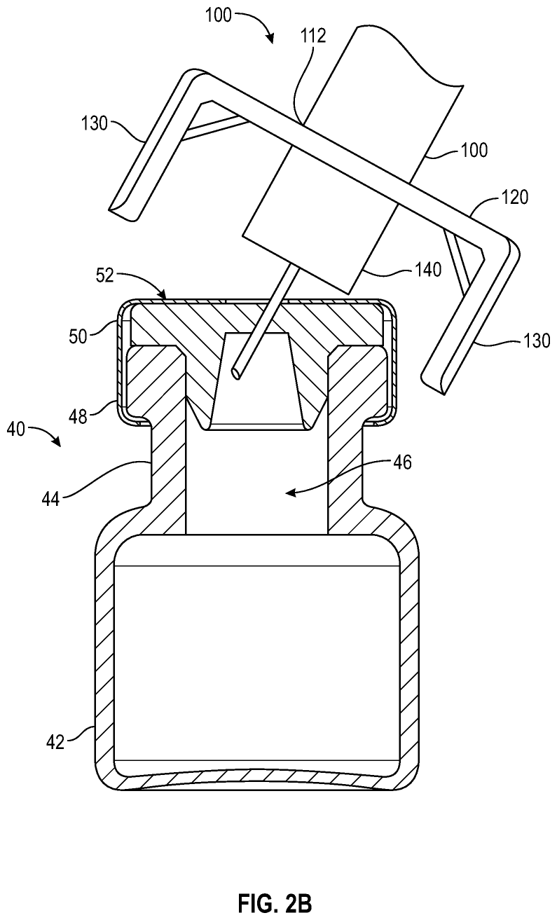

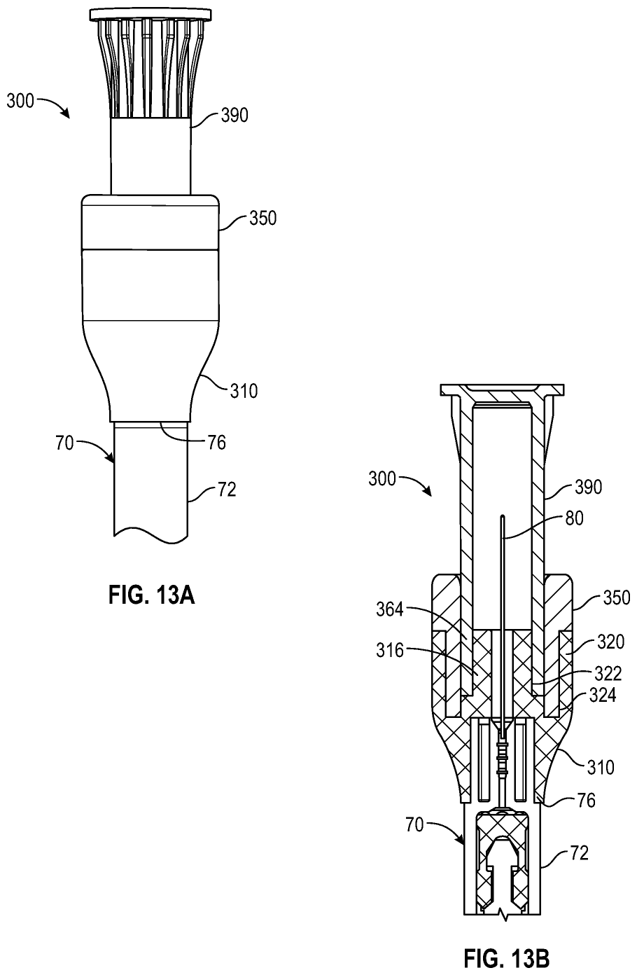

The vascular access device 100 further comprises a distal protrusion 140 extending distally from the flange 120, the distal protrusion 140 having a distal surface 144. The distal protrusion 140 in some embodiments has a cylindrical body and has the same width as the base 110. In some embodiments, as best shown in FIG. 3B, the base 110 and distal protrusion 140 share a common central axis C. As best shown in FIG. 2A, the base 110 and distal protrusion 140 share an aperture 142 extending through the base 110 and distal protrusion 140 for receiving a needle cannula 80. In some embodiments, the needle cannula 80 is affixed to the distal end 76 of the barrel 72 (as best shown in FIGS. 12 and 13B). As the vascular access device 100 is connected to the distal end 76 of the barrel, the needle cannula 80 extends from the distal end 76 of the barrel 72 past the distal surface 144 of the distal protrusion 140 of the vascular access device 100. In some embodiments, the needle cannula 80 extends a distance Dc from the distal surface 144 of the distal protrusion 140 of the vascular access device 100. In some embodiments, the distance Dc is sufficient to penetrate the rubber stopper 50 of the vial 40. As best shown in FIGS. 2A, 2B, 3A and 3B, in some embodiments, the distance Dc is sufficient to penetrate the rubber stopper 50 of the vial 40 at an angle having a range between 90 to 45 degrees relative to the rubber stopper 50 of the vial 40. In some embodiments, the distance Dc is 6 mm such that the needle cannula 80 can penetrate the rubber stopper 50 of the vial 40 at an angle having a range between 90 to 45 degrees relative to the rubber stopper 50 of the vial, where the vial 40 is a common or standard insulin vial.

As best shown in FIG. 3B, the two prongs 130 are spaced a distance Dp from each other. The distance Dp is greater than the width Wv (in cross-section) of the rubber stopper 50 of the vial 40 such that the two prongs 130 can pass over the rubber stopper 50 of the vial 40. Due to the two prongs 130 being passable over the rubber stopper 50 of the vial 40, the needle cannula 80 is capable of being fully inserted into the rubber stopper 50 of the vial 40 until the distal surface 144 of the protrusion 140 abuts the top surface 52 of the rubber stopper 50 to withdraw medicament from the vial 40.

FIG. 2B illustrates that the needle cannula 80 and vascular access device 100 can be inserted at a 45-degree angle and at said angle the needle cannula 80 is in fluid communication with the neck 44 of the vial 40. FIG. 3B illustrates the range of motion of the needle cannula 80 and vascular access device 100 after the needle cannula 80. In particular, the needle cannula 80 and vascular access device 100 can access the top surface 52 of rubber stopper 50 at a range of 45 degrees to 135 degrees relative to the top surface 52 of rubber stopper 50. For the entire range of 45 degrees to 135 degrees relative to the top surface 52 of rubber stopper 50, the needle cannula 80 is in fluid communication with the neck 44 of the vial 40.

FIG. 3B illustrates the needle cannula 80 and vascular access device 100 inserted into the top surface 52 of rubber stopper 50 at a 90-degree angle relative to the top surface 52 of rubber stopper 50. In FIG. 3B, the distal surface 144 of the protrusion 140 fully contacts the top surface 52 of rubber stopper 50, and thus the entire distance Dc of needle cannula 80 is inserted through the top surface 52 of rubber stopper 50.

FIG. 4 illustrates the needle cannula 80 and vascular access device 100 partially inserted into the subcutaneous layer 26 of the skin 20 of a patient. As shown in FIG. 4, the U-shape of the flange 120 and two prongs 130 prevents full insertion of the needle cannula 80 into the skin 20 of a patient because the U-shape of the flange 120 and two prongs 130 prevents the epidermis layer 22 from contacting the distal surface 144 of the protrusion 140. Thus, the flange 120 and two prongs 130 effectively limit the distance Dc of needle cannula 80 as the vascular access device 100 is advanced against a flat surface such as the skin 20 of a patient.

As shown in FIG. 4, the two prongs 130 has a distance Df which extends a distance Dn beyond the distal surface 144 of the protrusion 140. Thus, the two prongs 130 are effective to limit the distance Dc of needle cannula 80 from fully entering the skin 20 of a patient by the distance Dn. In some embodiments, where the distance Dc of the needle cannula 80 is 6 mm, the distance Dn (the distance which the two prongs 130 extend beyond the distal surface 144 of the protrusion 140) is 2 mm, ensuring a 4 mm effective needle cannula length Dn′. In such a configuration, as shown in FIG. 4, the needle cannula 80 can only be inserted 4 mm into the skin 20 of a patient, thus being inserted only into the subcutaneous layer 26. Stated differently, the two prongs 130 are configured as a hard stop for preventing further insertion of the needle cannula into the skin 20 of a patient. Furthermore, the flange 120 and two prongs 130 ensure perpendicularity of the needle cannula 80 while administering the medicament (while inserting the needle cannula into the skin 20 of a patient).

A method of use of the vascular access device 100 comprises the steps of: attaching the proximal end 112 of the base 110 of the vascular access device 100 onto the distal end 76 of the syringe 70, inserting the needle cannula 80 into the top surface 52 of rubber stopper 50 until the distal surface 144 of the protrusion 140 abuts the top surface 52 of rubber stopper 50 of the vial 40, withdrawing medicament from the vial 40 by at least partially withdrawing the plunger rod 74 from the barrel 72 of the syringe 70, inserting the needle cannula 80 into the skin 20 of a patient until the two prongs 130 abut the epidermis layer 22 of the skin 20 of a patient, injecting medicament into the skin 20 of a patient by at least partially advancing the plunger rod 74 into the barrel 72 of the syringe 70.

As shown in FIGS. 5-10, the vascular access device 200 in accordance with one or more embodiments comprises a cylindrical base 210 having a proximal end 212 removably attached or integral to the distal end 76 of the syringe, and a distal end 214. The distal end 214 further comprises a flange 220 extending outward from the distal end 214. Each of the flange 220 further comprises a prong 230 extending distally from the flange 220. Each prong 230 has a distal end 232 extending a length from the flange 220. In some embodiments, the distal end 232 is a flat surface. In some embodiments, each prong 230 is at a substantially right angle relative to the flange 220 forming a U-shape with the flange 220. In some embodiments, each prong 230 is at an acute angle relative to the flange 220 forming a U-shape with the flange 220.

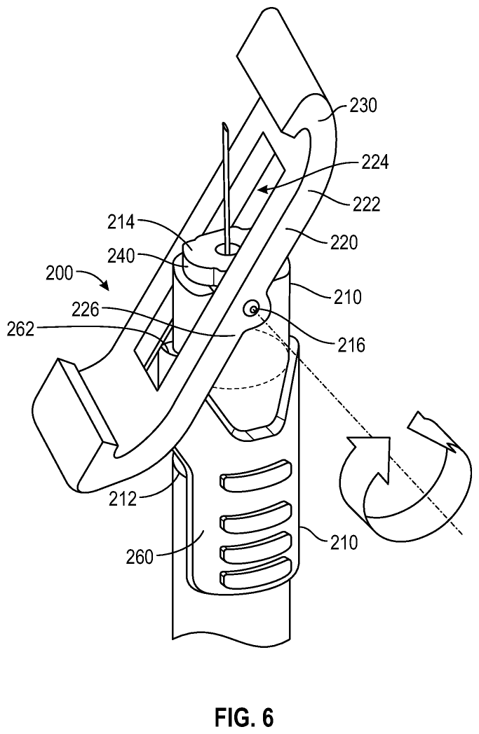

As best shown in FIG. 6, the flange 220 are connected by a body 222 between the flange 220. In some embodiments, the body 222 further comprises a cavity 224 for receiving a portion of the distal end 214 of the base 210. In some embodiments, the body 222 further comprises connection points 226 for connecting to a pivot 216 located at the distal end 214 of the base 210. In some embodiments, the pivot 216 is a pair of tabs to which the connection points 226 connect to. In some embodiments, the connection points 226 are apertures within the body 222 for receiving the pair of tabs of the pivot 216 of the base 210. In some embodiments, the base 210 and the body 222 of the flange 220 are two separate components which are snap-fit together by the pivot 216 and connection points 226. In some embodiments, the base 210 and the body 222 are of a unitary construction with a living hinge to provide a range of motion of the body 222 of the flange 220 relative to the base 210. Both the pivot 216-connection points 226 and the living hinge have an angular range of motion represented in FIG. 6 as Θ of 0 degrees to 75 degrees.

The vascular access device 200 further comprises a distal protrusion 240 extending distally from the flange 220, the distal protrusion 240 having a distal surface 244. The distal protrusion 240 in some embodiments has a cylindrical body and has the same width as the base 210. In some embodiments, the distal protrusion 240 has a width less than the width of the cavity 224 of the flange 220 such that the distal protrusion 240 can extend beyond the cavity 224 as shown in at least FIG. 6.

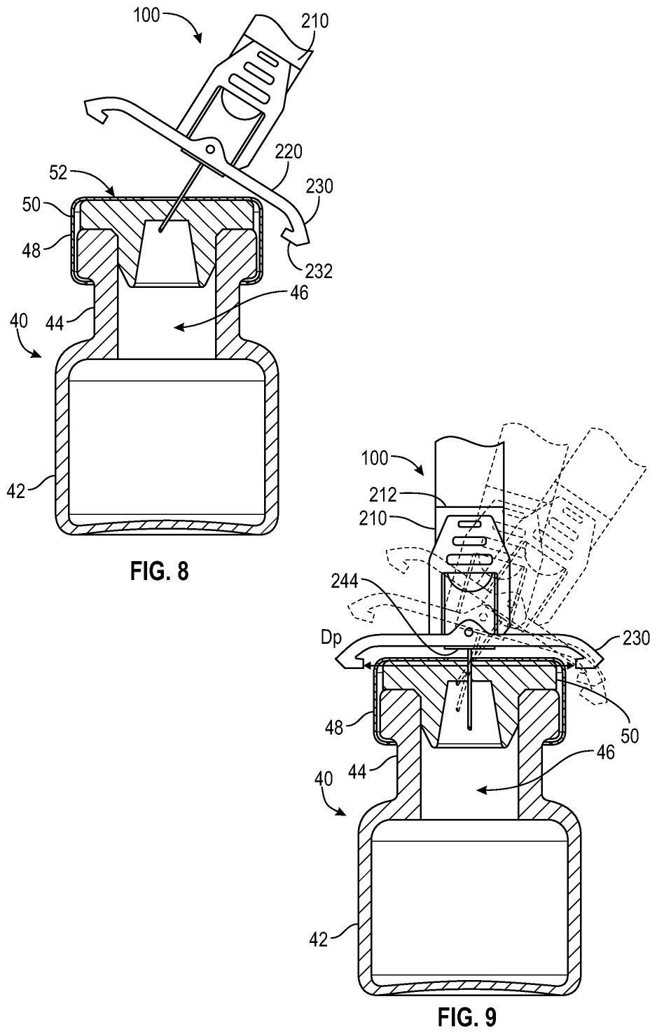

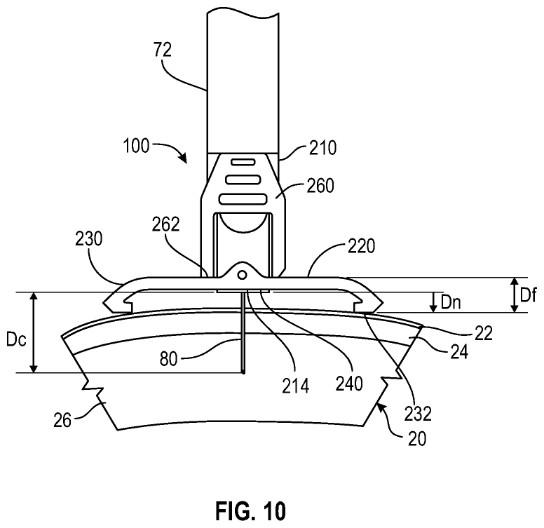

As best shown in FIG. 10, the needle cannula 80 extends a distance Dc from the distal surface 244 of the distal protrusion 240 of the vascular access device 100. In some embodiments, the distance Dc is sufficient to penetrate the rubber stopper 50 of the vial 40. As best shown in FIGS. 8-10, in some embodiments, the distance Dc is sufficient to penetrate the rubber stopper 50 of the vial 40 at an angle having a range between 90 to 45 degrees relative to the rubber stopper 50 of the vial 40 due to rotation of the flange 220 rotating about the pivot 216. In some embodiments, the distance Dc is 6 mm such that the needle cannula 80 can penetrate the rubber stopper 50 of the vial 40 at an angle having a range between 90 to 45 degrees relative to the rubber stopper 50 of the vial 40, where the vial 40 is a common or standard insulin vial.

As best shown in FIG. 9, the two prongs 230 are positioned a distance Dp from each other. The distance Dp is greater than the width Wv of the rubber stopper 50 of the vial 40 such that the prongs 230 can pass over the rubber stopper 50 of the vial 40. Due to the prong 230 being passable over the rubber stopper 50 of the vial 40, the needle cannula 80 is capable of being fully inserted into the rubber stopper 50 of the vial 40 until the distal surface 244 of the protrusion 240 abuts the top surface 52 of the rubber stopper 50 to withdraw medicament from the vial 40.

FIGS. 8 and 9 illustrate that the needle cannula 80 and vascular access device 200 can be inserted at a 45-degree angle and at said angle the needle cannula 80 is in fluid communication with the neck 44 of the vial 40. FIG. 9 illustrates the range of motion of the needle cannula 80 and vascular access device 200 after the needle cannula 80. In particular, the needle cannula 80 and vascular access device 200 can access the top surface 52 of rubber stopper 50 at a range of 45 degrees to 135 degrees relative to the top surface 52 of rubber stopper 50. For the entire range of 45 degrees to 135 degrees relative to the top surface 52 of rubber stopper 50, the needle cannula 80 is in fluid communication with the neck 44 of the vial 40. In FIG. 9, the distal surface 244 of the protrusion 240 fully contacts the top surface 52 of rubber stopper 50, and thus the entire distance Dc of needle cannula 80 is inserted through the top surface 52 of rubber stopper 50.

FIG. 10 illustrates the needle cannula 80 and vascular access device 200 partially inserted into the subcutaneous layer 26 of the skin 20 of a patient. As shown in FIG. 10 the U-shape of the flange 220 prevents full insertion of the needle cannula 80 into the skin 20 of a patient because the U-shape of the flange 220 prevents the epidermis layer 22 from contacting the distal surface 244 of the protrusion 240. Thus, the flange 220 and prongs 230 effectively limit the distance Dc of needle cannula 80.

As shown in FIG. 10, the prongs 230 have a distance Df which extends a distance Dn beyond the distal surface 244 of the protrusion 240. Thus, the prong 230 are effective to limit the distance Dc of needle cannula 80 from fully entering the skin 20 of a patient by the distance Dn. In some embodiments, where the distance Dc of the needle cannula 80 is 6 mm, the distance Dn (the distance which the prongs 230 extend beyond the distal surface 244 of the protrusion 240) is 2 mm, ensuring a 4 mm effective needle cannula length Dn′. In such a configuration, as shown in FIG. 10 the needle cannula 80 can only be inserted 4 mm into the skin 20 of a patient, thus being inserted only into the subcutaneous layer 26. Stated differently, the prongs 230 are configured as a hard stop for preventing further insertion of the needle cannula 80 into the skin 20 of a patient. Furthermore, the flange 220 and prongs 230 ensure perpendicularity of the needle cannula 80 while administering the medicament (while inserting the needle cannula into the skin 20 of a patient).

In some embodiments, as shown in FIGS. 6 and 10, the vascular access device 200 further comprises a slide 260 which surrounds the base 210 and is advanceable both proximally and distally relative to the base 210. The slide 260 includes a distal end 262 having a width greater than the cavity 224 of the flange 220. Advancement of the slide 260 in the distal direction causes the distal end 262 of the slide 260 to abut the flange 220, causing the flange 220 to become perpendicular to the distal end 262 and the base 210. FIG. 6 illustrates a retracted position of the slide 260 in which the flange 220 can freely pivot and FIG. 10 illustrates a fully advanced position of the slide 260 in which the distal end 262 of the slide 260 has fully abutted the flange 220.

In embodiments not having the slide 260, the flange 220 will passively adjust to become perpendicular to the needle cannula 80 and base 110. In particular, if the flange 220 is not perpendicular to the needle cannula 80 and base 110 during insertion, the prong 230 distal to the other prong 230 (due to the acute angle) will come into contact with the skin of a patient first, resulting in rotation of the flange 220 until the other prong 230 also comes into contact with the skin of a patient. Stated differently, if the flange 220 is not at a right angle relative to the skin, insertion of the vascular access device 200 will cause the flange 220 to become at a right angle as the forward prong 230 will adjust the entire flange 220 when it comes into contact with skin of a patient.

A method of use of the vascular access device 200 comprises the steps of: attaching the proximal end 212 of the base 210 of the vascular access device 200 onto the distal end 76 of the syringe 70, inserting the needle cannula 80 into the top surface 52 of rubber stopper 50 until the distal surface 244 of the protrusion 240 abuts the top surface 52 of rubber stopper 50 of the vial 40, withdrawing medicament from the vial 40 by at least partially withdrawing the plunger rod 74 from the barrel 72 of the syringe 70, inserting the needle cannula 80 into the skin 20 of a patient until the prong 230 abut the epidermis layer 22 of the skin 20 of a patient, injecting medicament into the skin 20 of a patient by at least partially advancing the plunger rod 74 into the barrel 72 of the syringe 70.

As shown in FIGS. 11-21B, the vascular access device 300 in accordance with one or more embodiments comprises a cylindrical base 310 and a compressible foam tip 350. FIG. 11 illustrates an assembled view of the vascular access device 300 onto the syringe 70 and FIG. 12 illustrates an exploded view of the vascular access device. As shown in FIGS. 11 and 12, in some embodiments the vascular access device further includes a sterile cap 390.

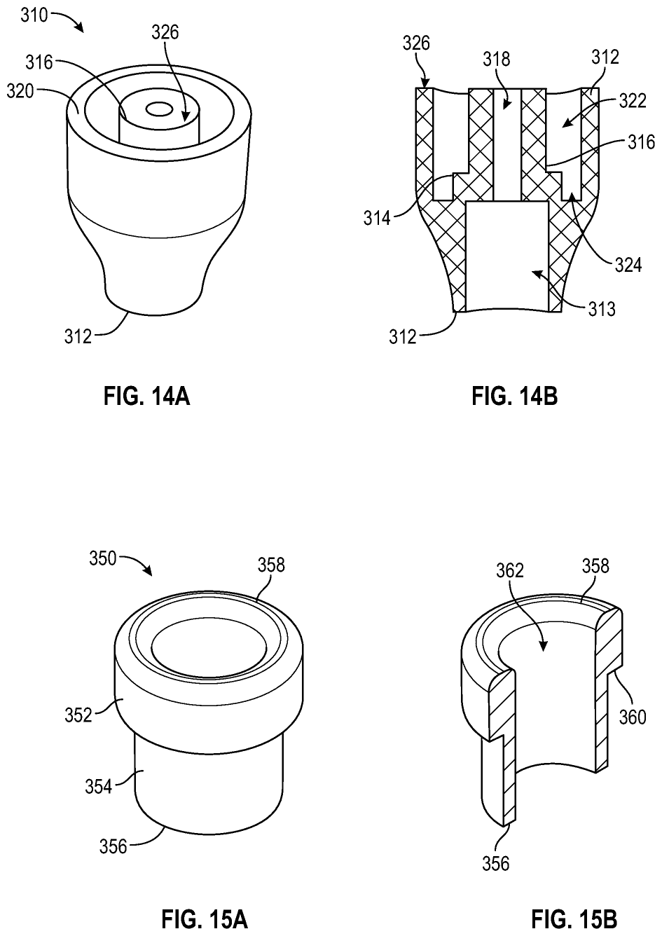

As shown in FIGS. 13A through 15B, the base 310 has a cylindrical shape comprising a proximal end 312 and a distal end 314. The proximal end 312 comprises a cavity 313 for receiving the distal end 76 of the syringe 70. From the distal end 314 extends an elongate tip 316 in a distal direction. The elongate tip 316 includes aperture 318 extending through the elongate tip and the base 310 for receiving the needle cannula 80 (as best shown in FIGS. 13B and 14B). The base 310 further comprises a collar 320 extending from the distal end 314, the collar 320 surrounding the elongate tip 316 and the aperture 318. As shown in FIGS. 14A and 14B, the collar 320 is spaced a distance from the elongate tip 316 such that a channel 322 is formed between the collar 320 and the elongate tip 316. In some embodiments, the elongate tip 316 and collar 320 extend the same length and share a common distal surface 326. In some embodiments, the base further comprises a glue channel 324 below the distal end 314 of the base and extending partially within the channel 322. As explained in further detail below, the glue channel 324 can be filled with medical-grade adhesive for non-removably securing the compressible foam tip 350 onto the base 310.

The compressible foam tip 350 has a substantially cylindrical shape. The compressible foam tip 350 has a distal portion 352 and a proximal portion 354, the distal portion 352 having a larger diameter than a diameter of the proximal portion 354. The compressible foam tip 350 has a proximal end 356, a distal end 358 and a contact surface 360. The contact surface 360 is formed by a proximal ridge of the distal portion 352 due to the distal portion 352 having a larger diameter than a diameter of the proximal portion 354. The compressible foam tip 350 is hollow, having an aperture 362 extending therethrough. In some embodiments, the distal end 358 is rounded.

As best shown in FIG. 13B, the glue channel 324 is lateral to the channel 322. The compressible foam tip 350 is inserted and affixed to the base 310 until the distal surface 326 of the base 310 abuts the contact surface 360 of the compressible foam tip 350 and the proximal end 356 of the compressible foam tip 350 is fully inserted into the glue channel 324. As shown, the proximal end 356 of the compressible foam tip 350 has a substantially equal width with the glue channel 324. Due to the glue channel 324 being lateral to the channel 322, a cavity 364 is formed between the elongate tip 316 of the base 310 and the proximal portion 354 of the compressible foam tip 350. The cavity 364 can receive the sterile cap 390.

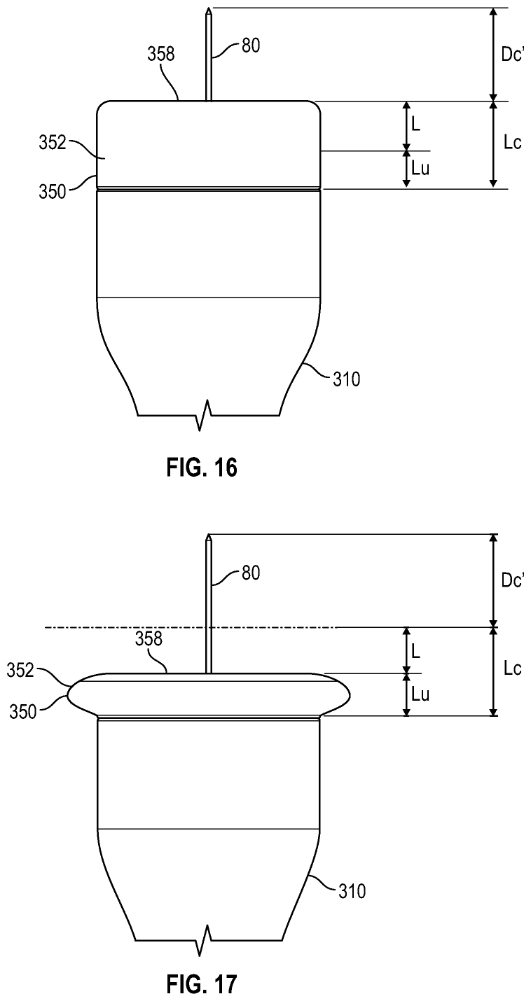

As best shown in FIGS. 16 and 17, the distal portion 352 of the compressible foam tip 350 is compressible from an initial, uncompressed length Lu in an initial, uncompressed state to a final, compressed length Lc in a final, compressed state. The distal portion 352 compresses a length L from the uncompressed length Lu to the compressed length Lc by depressing the distal end 358 against a surface. Stated differently, depressing the distal portion 352 of the compressible foam tip 350 against a surface causes the distal portion 352 to compress from the initial, uncompressed length Lu to the final, compressed length Lc by a length L. Release of the distal portion 352 of the compressible foam tip 350 causes the distal portion 352 to return to a final, uncompressed length Lu. Thus, the distal portion 352 of the compressible foam tip 350 has an initial, uncompressed length Lu, a final, compressed length Lc an final uncompressed length Lu.

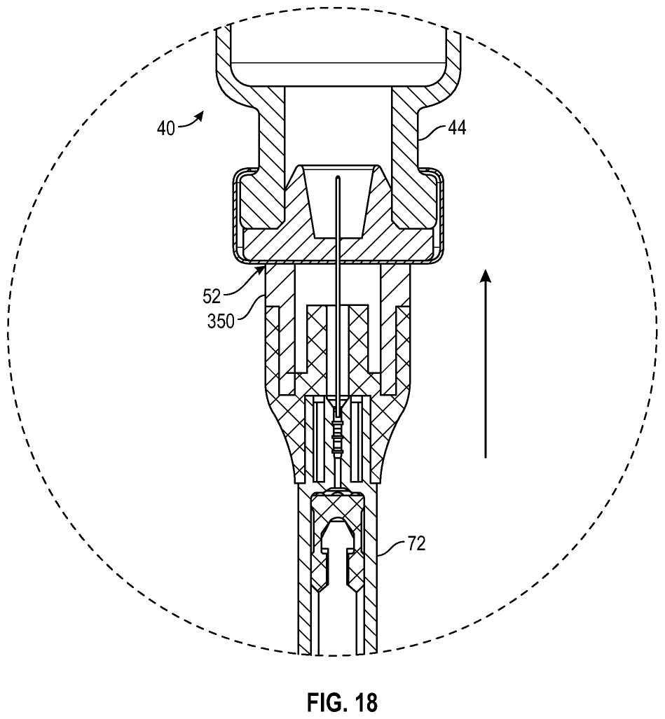

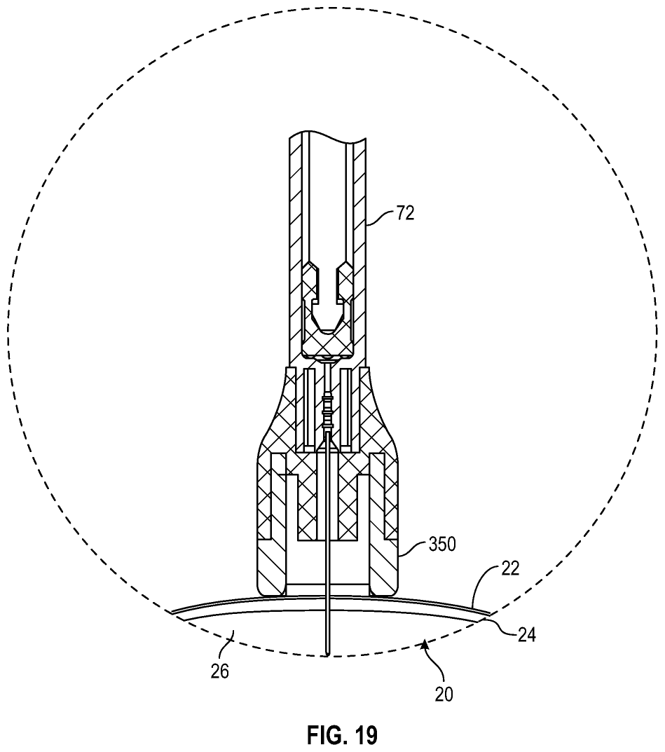

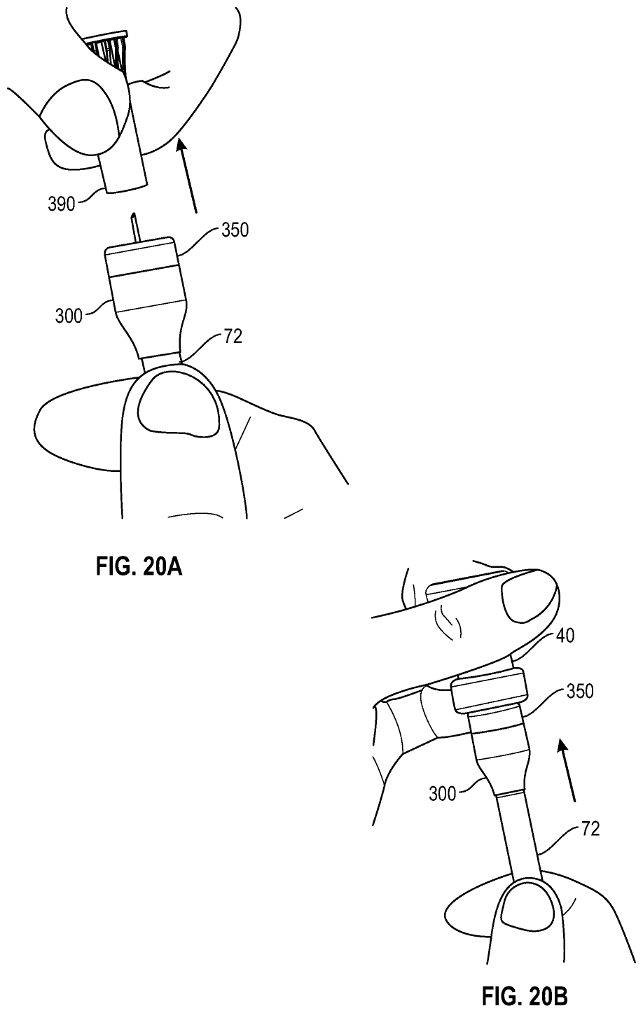

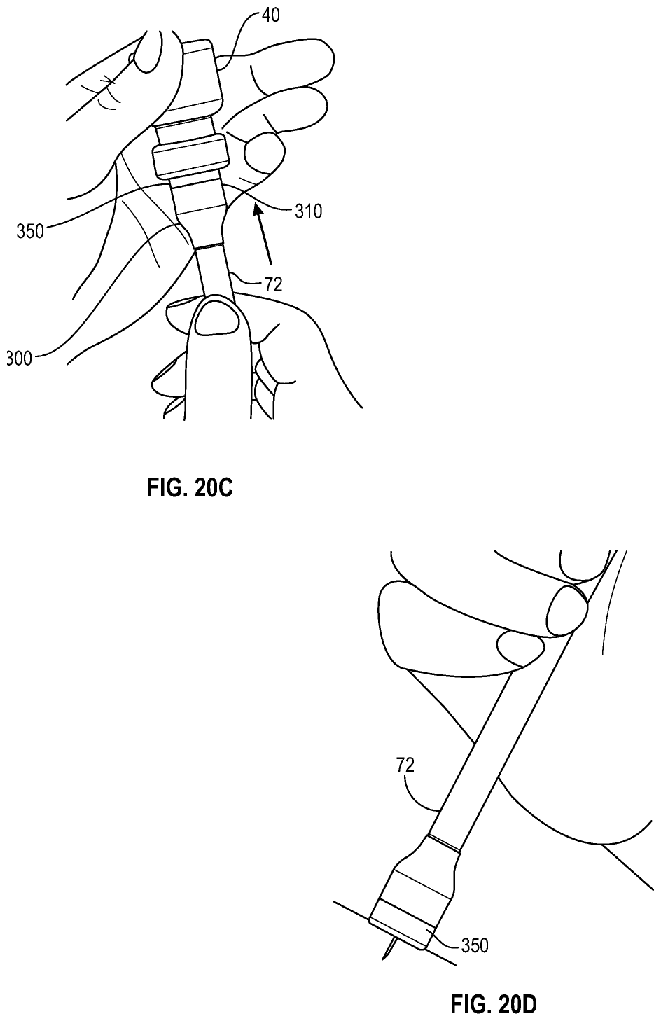

As best shown in FIGS. 16 and 17, the needle cannula 80 extends a distance Dc′ in the initial, uncompressed state and final uncompressed state. Compression of the compressible foam tip 350 to the final, compressed state results in the needle cannula 80 further extending by the length L in addition to the distance Dc′. As shown in FIGS. 18, and 20B, 20C, the compressible foam tip 350 is compressed by applying a compression force against the top surface 52 of the rubber stopper 50 such that the needle cannula 80 is inserted into the top surface 52 of the rubber stopper 50 by a combined length of distance Dc′ and length L. As shown in FIGS. 19 and 20D, the compressible foam tip 350 can abut the skin 20 of a patient by advancing the syringe 70 against the skin 20 of a patient by a force less than the compression force such that the compressible foam tip 350 does not deform and only the distance Dc′ is advanced into the skin 20 of a patient. Referring back to FIGS. 16 and 17, in some embodiments, where the distance Dc′ is 4 mm, and the length L is 2 mm, the effective needle length in the final, compressed state is 6 mm (the combined length of length Dc′ and length). In such a configuration, as shown in FIG. 16, 19, 20D, the needle cannula 80 can only be inserted 4 mm into the skin 20 of a patient, thus being inserted only into the subcutaneous layer 26.

As shown in FIGS. 20A through 20D, method of use of the vascular access device 300 comprises the steps of: attaching the proximal end 312 of the base 310 of the vascular access device 300 onto the distal end 76 of the syringe 70, inserting the needle cannula 80 into the top surface 52 of rubber stopper 50 until the distal end 358 of the distal portion 352 of the compressible foam tip 350 abuts the top surface 52 of rubber stopper 50 of the vial 40 as shown in FIG. 20B, further compressing the distal portion 352 against the top surface 52 of rubber stopper 50 of the vial 40 by the compression force, withdrawing medicament from the vial 40 by at least partially withdrawing the plunger rod 74 from the barrel 72 of the syringe 70, inserting the needle cannula 80 into the skin 20 of a patient until the distal end 358 of the distal portion 352 of the compressible foam tip 350 abuts the top surface 52 of rubber stopper 50 of the vial 40 as shown in FIG. 20D and. injecting medicament into the skin 20 of a patient by at least partially advancing the plunger rod 74 into the barrel 72 of the syringe 70.

As shown in FIGS. 22-25, the vascular access device 400 in accordance with one or more embodiments comprises a cylindrical base 410 having a distal end geometry 450 configured to limit effective needle cannula length. The base 410 includes aperture 418 extending through the base 410 for receiving the needle cannula 80. As shown in FIGS. 11 and 12, in some embodiments, the vascular access device further includes a sterile cap 490.

The base 410 has a cylindrical shape comprising a proximal end 412 and a distal end 414, the distal end having a distal end geometry 450 configured to limit effective needle cannula length. As best shown in FIG. 25, the distal end geometry 450 comprises a sloped surface 452 and a distally flat surface 454 located distal to the sloped surface 452. The sloped surface 452 is sloped at an angle Θ relative to the base 410. In some embodiments, the angle Θ is 45 degrees. In some embodiments, the angle Θ is 30 to 60 degrees. In some embodiments, the distally flat surface 454 includes a lateral extension 456.

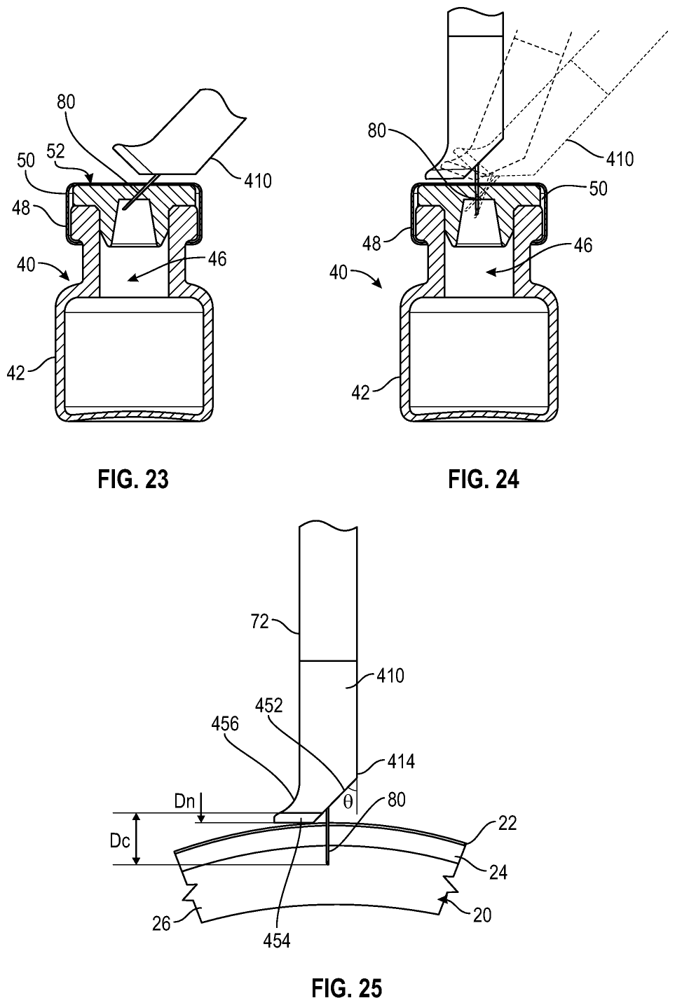

As best shown in FIGS. 23 and 24, the sloped surface 452 is configured to allow for the needle cannula 80 to be advanced at either the angle Θ or at a right angle relative to the top surface 52 of the rubber stopper 50.

As best shown in FIG. 25, the needle cannula 80 extends from the sloped surface 452 at the aperture 418 a distance Dc, and the distally flat surface 454 is located a distance Dn from the aperture 418. When the vascular access device 400 is inserted into the skin 20 of a patient, the distally flat surface 454 can prevent the needle cannula from being fully inserted. In particular, when the vascular access device 400 is inserted until the distally flat surface 454 abuts the epidermis layer 22 as shown in FIG. 25. In some embodiments, where the distance Dc is 6 mm, the distance Dn is 2 mm and thus the needle cannula 80 can only be inserted 4 mm. In some embodiments, the distance Dc is 6 mm such that the needle cannula 80 can penetrate the rubber stopper 50 of the vial 40 at an angle having a range between 90 degrees relative to the rubber stopper 50 of the vial 40 to 45 degrees rubber stopper 50 of the vial 40, where the vial 40 is a common or standard insulin vial.

While the present disclosure has been shown and described with reference to certain exemplary embodiments thereof, it will be understood by those skilled in the art that various changes in form and details may be made therein without departing from the spirit and scope of the embodiments of the present disclosure. Also, the inner and/or the outer housing of the disinfection cap can be single shot molded, or made by other suitable process. Furthermore, any of the features or elements of any exemplary implementations of the embodiments of the present disclosure as described above and illustrated in the drawing figures can be implemented individually or in any combination(s) as would be readily appreciated by skilled artisans without departing from the spirit and scope of the embodiments of the present disclosure.

In addition, the included drawing figures further describe non-limiting examples of implementations of certain exemplary embodiments of the present disclosure and aid in the description of technology associated therewith. Any specific or relative dimensions or measurements provided in the drawings other as noted above are exemplary and not intended to limit the scope or content of the inventive design or methodology as understood by artisans skilled in the relevant field of invention.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has provided a description with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A vascular access device comprising:
cylindrical base having a proximal end and a distal end;
a flange comprising two prongs extending distally from the flange, the two prongs having a distal end extending a length from the flange, the two prongs are placed a distance Dp from each other, the distance Dp being greater than a width Wv of a rubber stopper of a vial such that the two prongs can pass over the rubber stopper;
a distal protrusion extending distally from the flange, the distal protrusion having a distal surface and an aperture extending from the proximal end of the base to the distal surface of the distal protrusion; and,
a needle cannula extending through the aperture, the needle cannula extending a distance Dc from the distal surface of the distal protrusion;
wherein the two prongs extend a distance Dn from the distal surface of the distal protrusion.

2. The device of claim 1, wherein the proximal end of the cylindrical base is removably attached or integral to a distal end of a barrel of a syringe.

3. The device of claim 2, wherein the needle cannula is affixed to the distal end of the barrel extends from the distal end of the barrel of the syringe through the aperture.

4. The device of claim 1, wherein the two prongs are at a substantially right angle relative to the flange forming a U-shape with the flange.

5. The device of claim 1, wherein the distance Dc is sufficient to penetrate the rubber stopper of the vial.

6. The device of claim 5, wherein the distance Dc is 6 mm and the vial is an insulin vial.

7. The device of claim 6, wherein the needle cannula is sufficient to penetrate the insulin vial at an angle having a range between 90 to 45 degrees relative to the rubber stopper.

8. The device of claim 1, wherein the prongs are effective to limit the needle cannula from fully entering a skin of a patient by the distance Dn.

9. The device of claim 1, wherein the distance Dc of the needle cannula is 6 mm and the distance Dn is 2 mm, and where the two prongs limit contact of the distal surface of the distal end from contacting a flat surface, the cannula can only be inserted 4 mm into said flat surface.

10. The device of claim 1, wherein the flange is connected to the base by connection points of the flange for connecting the flange to a pivot located at the distal end of the base.

11. The device of claim 10, wherein the base and the flange are separate components which snap-fit together by a pivot of the flange and connection points of the distal end of the base.

12. The device of claim 10, wherein the pivot and connection points have an angular range of motion 0 degrees to 75 degrees.

13. The device of claim 10, wherein the base and the flange are of a unitary construction and connected by a living hinge to provide a range of motion of the base of the flange relative to the base.

14. A vascular access device comprising:
a cylindrical base having a proximal end and a distal end, the distal end having an elongate tip and a collar extending therefrom, the collar surrounding the elongate tip, the elongate tip having an aperture extending from the proximal end of the base to the elongate tip, the collar spaced a distance from the elongate tip such that a channel is formed between the collar and the elongate tip, the channel having a distal surface;
a compressible foam tip having a proximal end and a distal end, and a proximal portion and a distal portion, the distal portion having a larger diameter than a diameter of the proximal portion forming a contact surface, the compressible foam tip inserted and affixed to the base where the distal surface of the base abutting the contact surface of the compressible foam tip, the distal portion of the compressible foam tip is compressible from an initial uncompressed length Lu in an initial uncompressed state to a final compressed length Lc in a final compressed state; and,
a needle cannula extending through the aperture, the needle cannula extending a distance Dc′ from the distal portion in the uncompressed state;
wherein compression of the compressible foam tip to the final compressed state results in the needle cannula further extending by a length L in addition to the distance Dc′.

15. The device of claim 14, wherein the proximal end of the cylindrical base is removably attached or integral to a distal end of a barrel of a syringe.

16. The device of claim 15, wherein the needle cannula is affixed to the distal end of the barrel extends from the distal end of the barrel of the syringe through the aperture.

17. The device of claim 14, wherein the elongate tip and collar extend the same length and share the distal surface.

18. The device of claim 14, wherein the base further comprises a glue channel below the distal end of the base and at least partially within the channel, the glue channel lateral to the channel.

19. The device of claim 18, wherein the proximal end of the compressible foam tip is inserted into the glue channel, the proximal end of the compressible foam tip having a substantially equal width with the glue channel, wherein a cavity is formed between the elongate tip of the base and the proximal portion of the compressible foam tip.

20. The device of claim 14, wherein the compressible foam tip is compressed by applying a compression force against a top surface of a rubber stopper such that the needle cannula is inserted into the top surface of the rubber stopper by a combined length of distance Dc′ and length L.

21. The device of claim 14, wherein the distance Dc′ is 4 mm.

22. The device of claim 20, wherein the combined length of distance Dc′ and length L is 6 mm.

23. A vascular access device comprising:
a cylindrical base having a proximal end and a distal end, the distal end including a distal end geometry, the base including an aperture extending through the base, the distal end geometry having a sloped surface and a distally flat surface located distal to the sloped surface, the sloped surface is sloped at an angle Θ relative to the base, the distally flat surface located a distance Dn from the aperture; and,
a needle cannula extending through the aperture, needle cannula extending from the sloped surface at the aperture by a distance Dc.

24. The device of claim 23, wherein the distally flat surface includes a lateral extension.

25. The device of claim 23, wherein the distance Dc is 6 mm and the distance Dn is 2 mm, and the needle cannula has an effective length of 4 mm.
